(19) United States
(12) Patent Application Publication
(10) Pub. No.: US 2024/0389832 A1
(43) Pub. Date: Nov. 28, 2024

(54) AN ENDOSCOPY DEVICE

(71) Applicants: Singapore Health Services Pte Ltd, Singapore (SG); Singapore University of Technology and Design, Singapore (SG)

(72) Inventors: Chi Yuan Ian Loh, Singapore (SG); Wei Ming James Kwek, Singapore (SG); Tee Sin Lee, Singapore (SG); Jian Hui Kevin Wee, Singapore (SG); Yue Han John Tan, Singapore (SG); Jing Ting Bernice Kwok, Singapore (SG); Fang Sing Nicole Sim, Singapore (SG); Chi Yee Alysia Chai, Singapore (SG)

(21) Appl. No.: 18/690,714

(22) PCT Filed: Sep. 12, 2022
(86) PCT No.: PCT/SG22/50647

§ 371 Date: Mar. 8, 2024

(30) Foreign Application Priority Data
Sep. 10, 2021 (SG) 10202109976X

(51) Int. Cl. A61B 1/00; A61M 25/00

(52) U.S. Cl. CPC A61B 1/00066; A61B 1/0011; A61B 1/00135; A61M 2025/0004; A61M 25/0097

(57) ABSTRACT

An endoscopy device and a method of manufacturing an endoscopy device are disclosed. The endoscopy device comprises: a body having a handle portion and a housing portion, the housing portion having an outlet; a switching mechanism disposed in the housing portion; a rigid outer tube detachably coupled to the housing portion at the outlet, and a flexible inner tube extendable from the outer tube, wherein the switching mechanism is configured to retract or extend the inner tube outwardly through the outer tube.

### FIELD OF INVENTION

The present invention relates broadly, but not exclusively, to an endoscopy device and method of manufacturing the device.

### BACKGROUND

Typical ailments in the ear, nose and throat (ENT) region may include a throat infection, foreign objects lodged in the nose, ear or throat as well as growths in the said regions. Generally, an ENT patient will consult the nearest general practitioner (GP), usually in a primary care setting, when treating ENT conditions. However, GPs are neither equipped to visualise and diagnose conditions that are not in direct-line-of-sight, nor sufficiently equipped to perform procedures such as aural toiletting or removal of foreign objects. These patients would then have to be referred to an ENT specialist in a tertiary hospital for an examination or treatment under direct microscopy or nasoendoscopy. There are currently approximately 11.7 million ENT physicians around the world and ENT-related issues make up to 20% of adult consultations and 40% of paediatric consultations at a GP.

Current ENT endoscopes are bulky systems that would not fit in a GP's office as they often requiring dedicated consoles for the light and the video monitors respectively. In addition, current ENT endoscopes are too expensive to justify its use in a primary care setting in a GP clinic. Current ENT endoscopes are also highly specialized with many variants each for the ear, nose and throat. For example, ear endoscopes may include otoscopes for simple diagnostics and rigid endoscopes and massive microscopes for treatments, nose endoscopes may include rigid rhinoscopes or flexible rhinolaryngoscopes and throat endoscopes may include laryngoscope or rhinolaryngoscope. Many variants of ENT endoscopes thus exist, each being designed for a specific purpose. Rigid endoscopes may be used for shallow regions such as the ear or the entrance of the nose, flexible variants for hard-to-reach deeper parts of the nasal passage to the throat and each may come in many different sizes for diagnostic and therapeutic purposes.

There is currently no single portable solution for ENT diagnosis and simple treatment such as removal of foreign bodies, biopsy, suction and irrigation, which may lead to delayed patient diagnosis and treatment. Specialists are unable to bring their endoscopes to patients in wards due to the bulk of the light and video monitor consoles and GPs do not have an abundance of space in their clinics for all the specialised equipment an endoscope currently requires.

Accordingly, a need exists to provide a device that seeks to address some of the above problems. In particular, there is a need to design a dedicated primary care endoscope device to perform diagnosis and treatment and provide GPs, who have both the capacity and capability, in the primary care setting with such a device.

### SUMMARY

According to a first aspect of the present invention, there is provided an endoscopy device comprising: a body having a handle portion and a housing portion, the housing portion having an outlet; a switching mechanism disposed in the housing portion; a rigid outer tube detachably coupled to the housing portion at the outlet, and a flexible inner tube extendable from the outer tube, wherein the switching mechanism is configured to retract or extend the inner tube outwardly through the outer tube.

In an embodiment, the handle portion comprises means to control a distal end of the inner tube.

In an embodiment, the device further comprises at least one guide wire inserted inside the inner tube and coupled to a control button, wherein an activation of the button moves the distal end of the inner tube.

In an embodiment, the switching mechanism comprises a pulley to retract or extend the inner tube.

In an embodiment, the switching mechanism comprises a protrusion attached to the pulley, and wherein the housing portion comprises a slot to slidingly translate the protrusion to retract or extend the inner tube.

In an embodiment, the handle portion further comprises an inlet, and wherein the inlet is configured to receive a medical instrument.

According to a second aspect of the present invention, there is provided a method for manufacturing an endoscopy device, the method comprising: providing a body having a handle portion and a housing portion, the housing portion having an outlet; disposing a switching mechanism in the housing portion; detachably coupling a rigid outer tube to the housing portion at the outlet, and disposing a flexible inner tube in the outer tube, wherein the switching mechanism is configured to retract or extend the inner tube outwardly through the outer tube.

In an embodiment, the method further comprises inserting at least one guide wire inside the inner tube and coupling one end of the guide wire to the distal end of the inner tube and the other end of the guide wire to a control button, such that an activation of the button moves the distal end of the inner tube.

In an embodiment, disposing the switching mechanism in the housing portion comprises attaching a protrusion to the pulley and slidingly translating the protrusion to retract or extend the inner tube via a slot of the housing portion.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1A shows a perspective view of an endoscopy device, according to an example embodiment.

FIG. 1B shows a side view of the device of FIG. 1A, according to an example embodiment.

FIG. 2A shows a cross-sectional side view of the handle of the device, according to an example embodiment.

FIG. 2B shows a perspective view of the handle of FIG. 2A.

FIG. 3A shows a cross-sectional side view of the switching mechanism with the flexible inner tube extended long, according to an example embodiment.

FIG. 3B shows a cross-sectional side view of the switching mechanism with the flexible inner tube retracted short, within the rigid outer tube, according to an example embodiment.

FIG. 4 shows an exploded view of the switching mechanism of FIG. 3A, according to an alternative embodiment.

FIG. 5A shows a close up perspective view of the outer tube detached from the housing portion, according to an example embodiment.

FIG. 5B shows a close up perspective view of the outer tube attached to the housing portion, according to an example embodiment.

FIGS. 5C to 5E show side views of the outer tube in various positions with respect to the housing portion, according to an example embodiment.

FIG. 6A shows a side cross sectional view of a distal end control of the handle portion, according to an example embodiment.

FIG. 6B shows a close up side cross sectional view of a distal end of the inner tube, according to an example embodiment.

FIGS. 7A and 7B show side cross sectional views of a suction mechanism, according to an example embodiment.

FIG. 8 shows a flow chart illustrating a method for manufacturing an endoscopy device, according to the example embodiments.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description. Herein, an endoscopy device is presented in accordance with present embodiments having the advantages of allowing a single-physician operation by a General Practitioner (GP) in the primary care setting, enabling him/her to conduct diagnostic and therapeutic procedures in the ear, nose and throat with a single instrument that is portable, while maintaining the current capabilities of high-fidelity imaging, wireless data transmission, suctioning, and therapeutic instrumentation. The device may also provide interchangeability of the scope between long flexible and short rigid modes, with retractable scope functionality to cater to differing ear-nose-throat (ENT) regions and cavity depths. The endoscopy device may also be compatible with existing therapeutic instruments and provide added stability such that a single General Practitioner (GP), by using the device, can perform ENT procedures that are currently administered by ENT specialists.

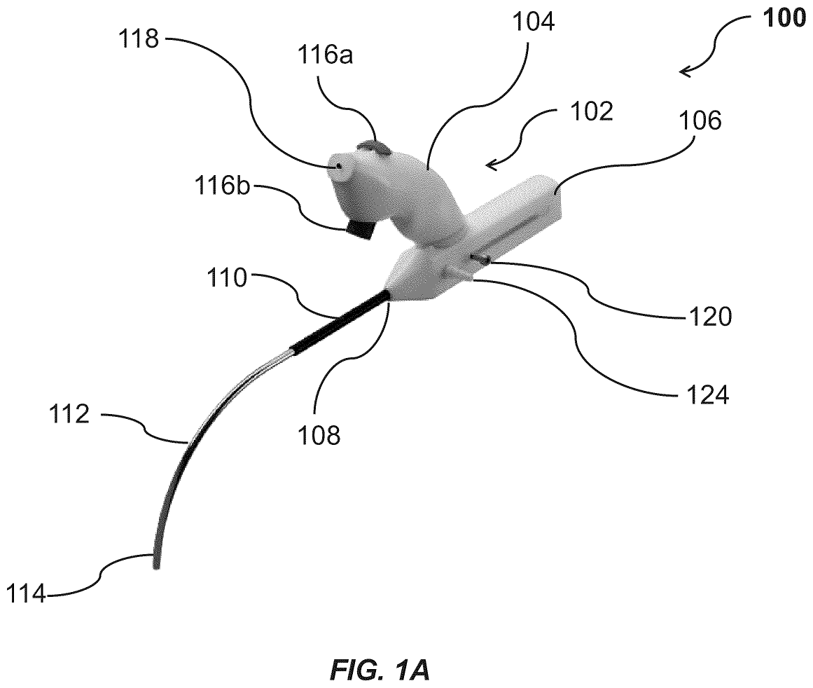

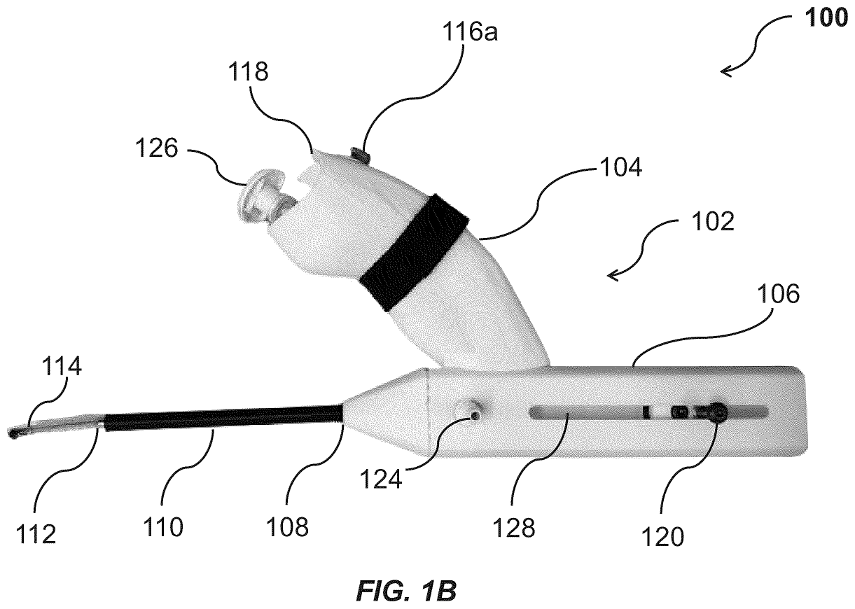

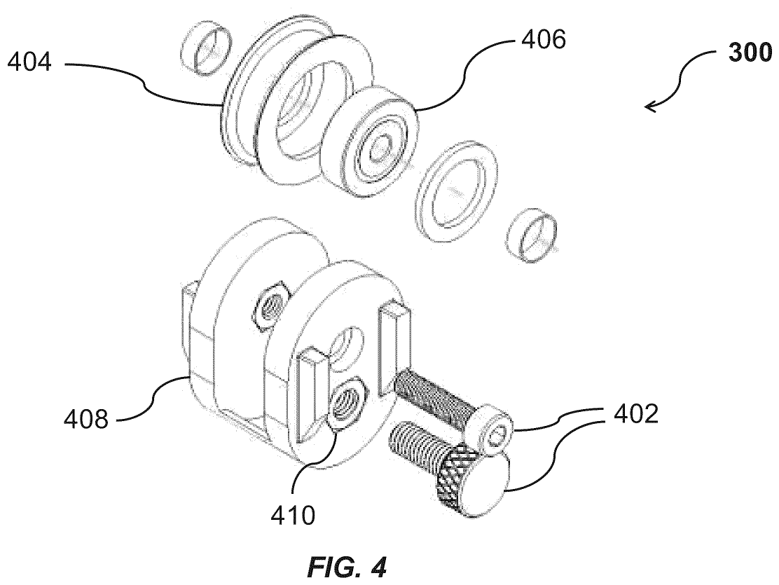

FIG. 1A shows a perspective view of an endoscopy device 100, according to an example embodiment. The device 100 comprises a body 102 having a handle portion 104 and a housing portion 106 having an outlet 108. The device 100 also includes a switching mechanism disposed in the housing portion 106 and a rigid outer tube 110 detachably coupled to the housing portion 106 at the outlet 108. The device 100 further includes a flexible inner tube 112 extendable from the outer tube 110 and the switching mechanism is configured to retract or extend the inner tube 112 outwardly through the rigid outer tube 110. The handle portion 104 may include means, such as an array of buttons 116a, 116b, to control a distal end 114 of the inner tube 112 and to control suctioning means respectively. The handle portion 104 may include an inlet port 118 to receive the inner tube 112. The switching mechanism may be a pulley system having a protrusion 120 attached to the pulley (as shown in FIG. 4) and protruding outwardly from the housing portion 106. The suction means may be disposed within the housing portion 106 and may include a suction port 124.

FIG. 1B shows a side view of the device 100 of FIG. 1A, according to an example embodiment. The handle portion 104 may include a controller 126 configured to maneuver the distal end 114 of the inner tube 112. The housing portion 106 may include a slot 128 to slidingly translate the protrusion 120 to retract or extend the inner tube 112.

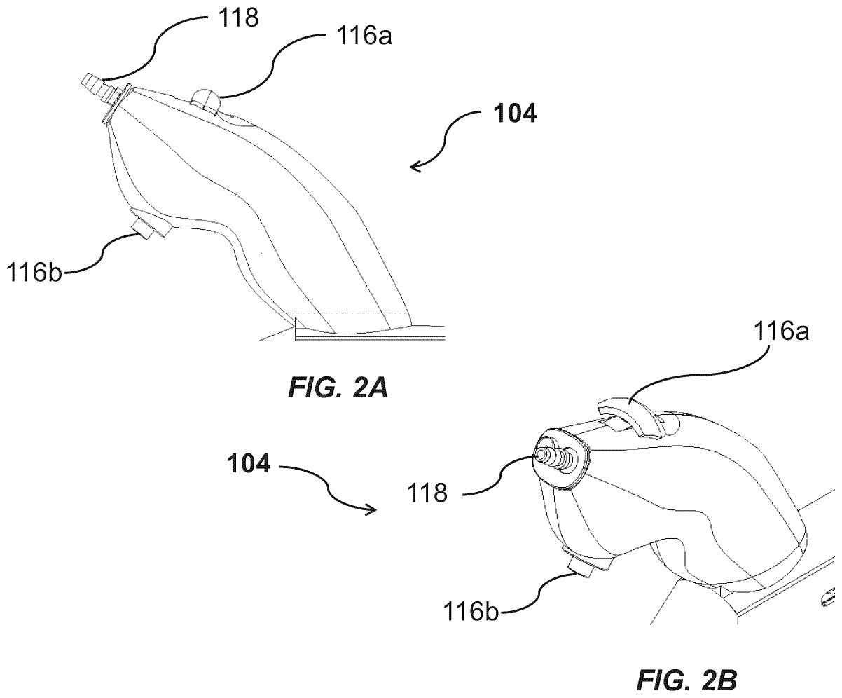

FIG. 2A shows a cross-sectional side view of the handle portion 104 of the device 100 while FIG. 2B shows a perspective view of the handle portion 104 of FIG. 2A, according to an example embodiment. The handle portion 104 may be adapted for use by a hand of a user and is designed to be operated by a single user. As shown in the Figures, the inlet port 118 may be located on a top surface of the handle portion 104. It can be appreciated that the inlet port 118 may be positioned in other surfaces of the handle portion 104 for ease of insertion of the inner tube 112. The handle portion 104 may also be configured to accommodate electronics associated with the inner tube 112. A control button 116a may control certain functions of the inner tube 112, such as the ejection of water or air for irrigation, and may also control the distal end 114 of the inner tube 112.

As GPs in a private clinic or polyclinic are lacking in manpower with no spare nurses readily available to assist a simple procedure, it is thus important that the device 100 provides confidence to the GP to handle even therapeutic procedures alone. The handle portion 104 may have a centre of gravity that can be positioned within the palm of the clinician with less than 1 cm deviation from the centre region of the user's palm. The single user may be able to conduct procedures with optimal performance without hiccups in endoscope itself, for example basic diagnosis can be conducted within 5 minutes. The handle portion 104 may include a working channel large enough, for example at least 2.2 mm in diameter, to fit therapeutic instruments. The working channel may be airtight to allow suctioning and irrigation, ensuring no leakage of air or water. The working channel may be required for basic treatment procedures, specifically suction, irrigation, use of forceps for removal of foreign objects and biopsy.

In an embodiment, the handle portion 104 may be designed such that the shape and location of the controls or array of buttons 116a, 116b are user-friendly and intuitive to the user. For example, the handle portion 104 may be held using the user's left hand and the thumb can comfortably rest upon the control button 116a for distal end control and the index finger can easily locate the suction control button 116b. The insertion port, or inlet port 118, for instruments may also be conveniently located at the top surface of the handle portion 104 so that it is easily accessible to the user. In an alternate embodiment, the inlet port 118 may be used to perform therapeutic procedures in addition to receiving the inner tube 112. For example, suction catheters, instruments for grasping and biopsy forceps may be attached to the inlet port 118. These instruments, when attached to the inlet port 118, may allow the user to perform therapeutic procedures such as viewing of the ENT regions, suction, irrigation, removal of foreign objects and biopsy. The protrusion 120 may act as a sliding pulley control for extension or retraction of the inner tube 112 and is located at the side of the housing portion 106 as it is part of the preparation phase, hence the protrusion 120 would not get into the way of the user's hands when performing procedures.

The device 100 may be capable for the physician to operate the inner tube 112 independently, with no external logistical assistance from another physician or healthcare professional during the performance of all diagnostic or therapeutic procedures. Examples of diagnostic procedures include the viewing of ENT regions, with the ability to distinguish or recognise the presence and identity of foreign bodies and growths while examples of therapeutic procedures include the removal of foreign bodies and growths with the use of forceps, suctioning and irrigation.

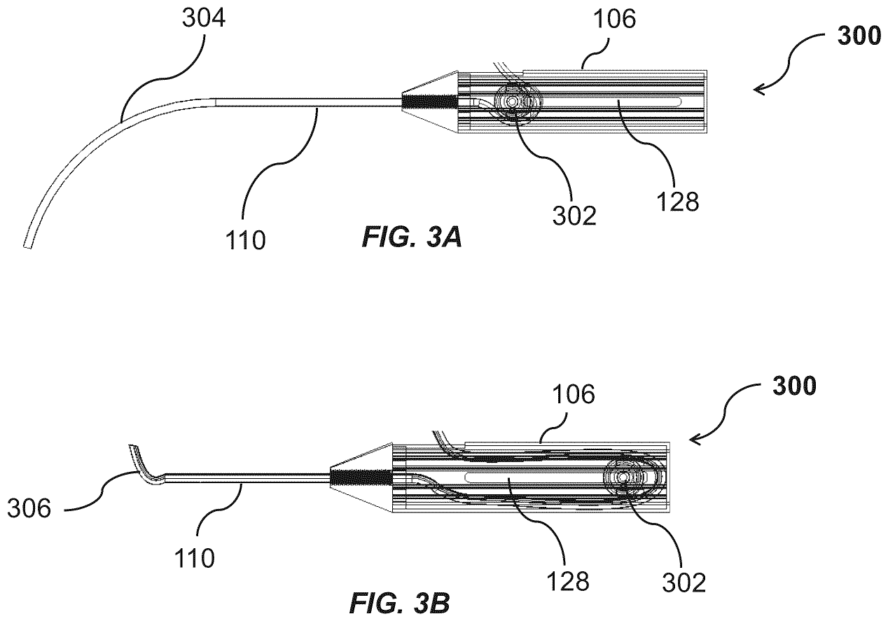

FIG. 3A shows a cross-sectional side view of the switching mechanism 300 with a flexible inner tube 304 extended long, according to an example embodiment. The switching mechanism 300 comprises a pulley 302 to retract or extend the inner tube 304 to interchange between a long-flexible inner tube or a short-rigid inner tube. In FIG. 3A, the long flexible inner tube 304 can be used to reach the back of the patient's throat to remove fish bones or viewing the sinus.

FIG. 3B shows a cross-sectional side view of the switching mechanism with a flexible inner tube 306 retracted short, within the rigid outer tube 110, according to an example embodiment. In FIG. 3B, the flexible inner tube 306 can allow for shallow diagnosis and treatment especially the ears. As the pulley 302 translates along the single axis of the slot 128, the inner tube 304, 306 shortens or lengthens out of the rigid outer tube 110 accordingly. For example, starting from FIG. 3A, when the pulley 302 is pulled further towards the right of the slot 128 to the position as shown in FIG. 3B. the inner tube 304 is shortened and vice versa.

FIG. 4 shows an exploded view of the switching mechanism 300 of FIG. 3A, according to an alternative embodiment. The switching mechanism 300 may follow the principles of a sliding pulley system, which can be made from three-dimensional printing and may include ball bearings and bolts 402 for assembly. The pulley 302 may be assembled with three-dimensional printed parts consisting of a wheel 404 having a grooved rim with a bearing 406 fitted in its inner circumference, a wheel housing 408, bolts 402 and nuts 410. The inner tube 112 is then inserted through the pulley 302, in the space between the wheel 404 and the housing 408. The switching mechanism 300 is then housed within a rectangular sliding compartment (as shown in FIG. 1B) and the pulley 302 can be manoeuvred externally via the protrusion 120 by sliding along the slot 129 of the housing portion 106.

The switching mechanism 300 can switch between the flexible and rigid modes under 3 minutes within the preparation phase. The preparation phase is where the inner tube 112 and additional instruments needed for diagnostic and therapeutic procedures are prepared beforehand, prior to the procedural steps. By using the device 100, the user may be able to retract the inner tube 112 with ease as the overall friction within the pulley system may be less than 2N and the deflection of the distal end 114 may be in a range of 240°.

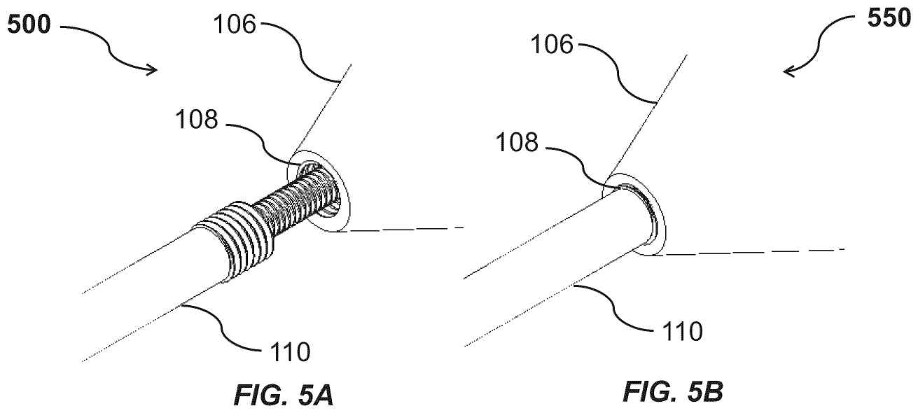

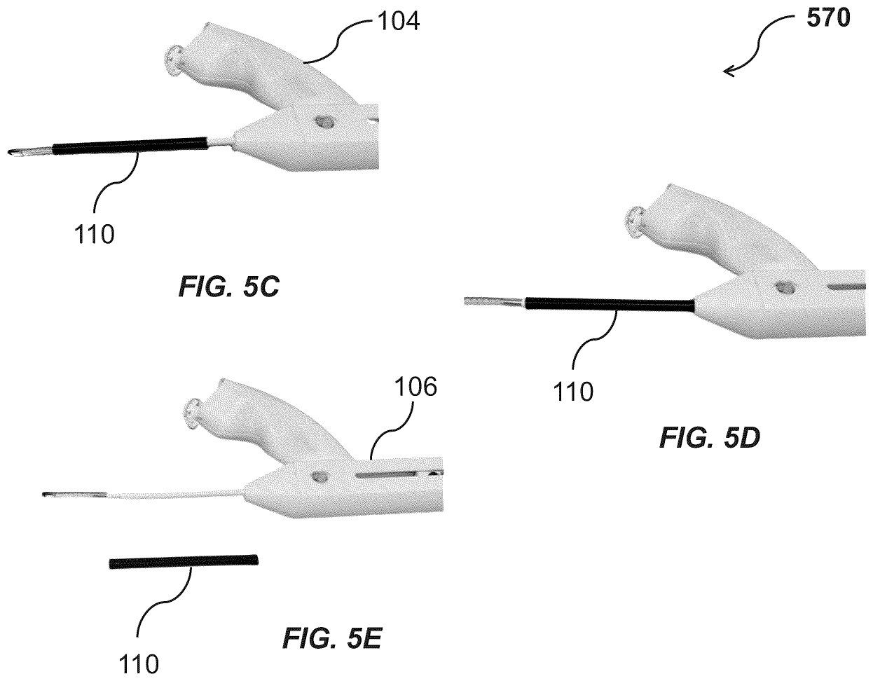

FIG. 5A shows a close up perspective view 500 of the outer tube 110 detached from the housing portion 106 while FIG. 5B shows a close up perspective view 550 of the outer tube 110 attached to the housing portion 106, according to an example embodiment. As shown in the Figures, the rigid outer tube 110 is made detachable from the housing portion 106 for the purpose of easy cleaning and easy manipulation of the flexible inner tube 112 in the case where more flexibility is required. This may be achieved by having fastening means, such as a screw and thread as shown. It can be appreciated that other fastening means are possible to attach and detach the outer tube 110 from the housing portion 106. FIGS. 5C to 5E show side views 570 of the outer tube 110 in various positions with respect to the housing portion 106. In an alternative embodiment, the rigid outer tube 110 may be integrated with the housing portion 106 as an extrusion from the outlet 108.

The rigid outer tube 110 may act as a short rigid inner tube 306 (as shown in FIG. 3B) to be used for shallower ENT regions and to provide additional control to the user to perform procedures in the rigid mode. It may also act as an anchor and a form of support for the flexible inner tube 112 to support the user in performing procedures alone, while preserving the function of the inner tube 112, as it is able to be inserted into passageways along with the inner tube 112. The device 100 and the rigid outer tube 110 may be designed in the form of Computer Aided Diagrams and may be three-dimensionally printed using Polylactic Acid (PLA) filament.

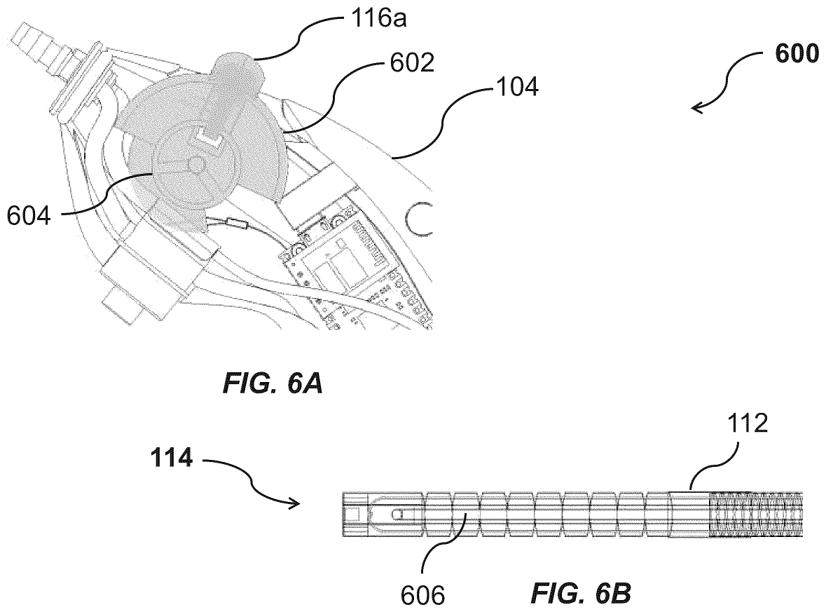

FIG. 6A shows a side cross sectional view 600 of a distal end control 602 of the handle portion 104 while FIG. 6B shows a close up side cross sectional view of a distal end 114 of the inner tube 112, according to an example embodiment. The distal end control 602 may include at least one guide wire 606 inserted inside the inner tube 112 and coupled to a control button 116a, wherein an activation of the control button 116a moves a distal end 114 of the inner tube 112. This can allow for manipulation of the distal end 114 in a single plane as the guide wires 606 are being pulled. At a proximal end of the inner tube 112, guide wires 606 may run through the length of the top and bottom of the inner tube 112 and secured at the distal end 114, as shown in FIG. 6B. The guide wires 606 are attached to a pulley 604, with each end of the guide wires 606 to opposite ends of the pulley 604. The pulley 604 is then anchored to the handle portion 104 and may be rotated forwards and backwards with the pulley 604 in line with the plane, as shown in FIG. 6B. For example, a rotation of the pulley forward will push the top guide wire and pull the bottom guide wire, resulting in the distal end 114 pointing downwards and vice versa.

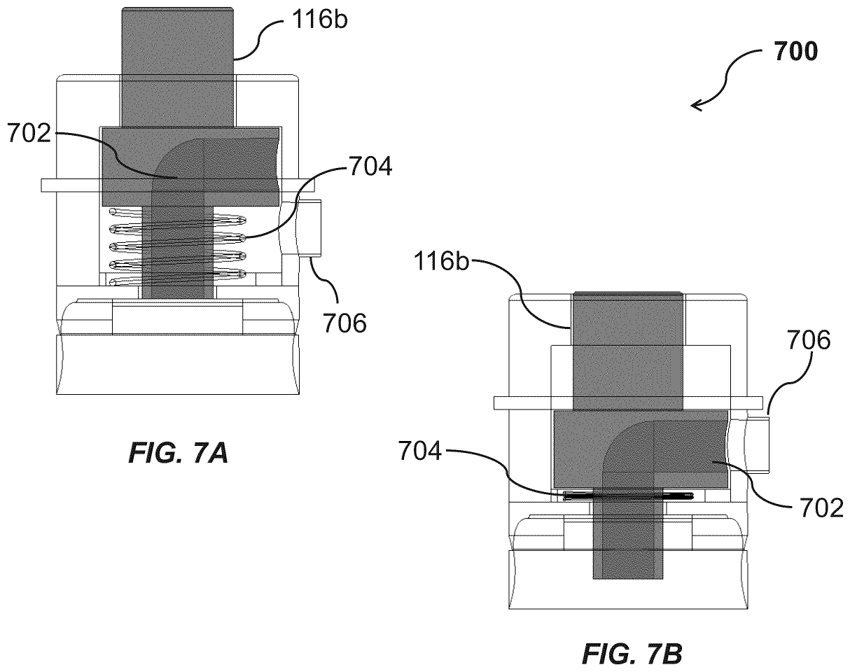

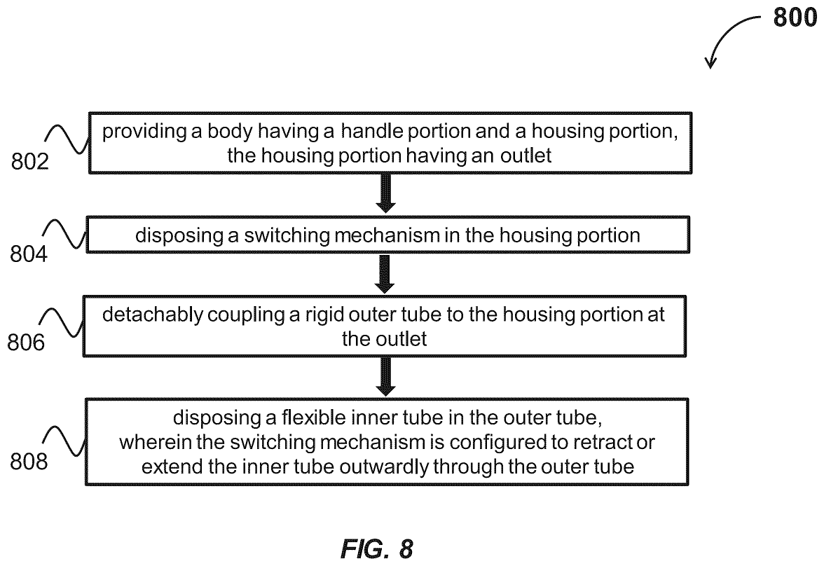

FIGS. 7A and 7B show side cross sectional views of a suction mechanism 700, according to an example embodiment. The suctioning mechanism 700 may include a port pathway 702 with a valve 704 that leads into the pathway of the working channel within the inner tube 112. The suction mechanism may be attached to a suction port 706 external to the handle portion 104 and the suction control button 116b can be pressed to open the valve 704 such that the suction mechanism 700 is activated through the working channel.

In an example implementation, a major component within the endoscope device 100 may be the control of the inner tube 112 at the distal end 114. As there would be repeated bending of the inner tube 112 due to distal end control 602 and manoeuvring of the inner tube as a whole within the patients' ENT regions, the safety factor and fatigue strength of the inner tube 112 does not reach its fatigue limit. The force required to push the distal end pulley 604 control a maximum to each side is estimated to be 3N. The material of the core of the inner tube 112 is taken to be ASTM A36 Steel. The dimensions required for calculations are as follows: Radius of the pulley=0.0215 m, radius of the insertion tube=0.00275 m, yield strength of ASTM A36 Steel=250 MPa (Engineering ToolBox, 2003), ultimate tensile strength of ASTM A36 Steel=400 MPa (Engineering ToolBox, 2003), moments, M=Fd=3×0.0215=0.0645 Nm. Assuming that the moments required to cause deflection at the distal end 114 is directly translated from the pulley control, For circular cross-sectional area,
Moment of inertia,

$$I = \frac{\text{?}}{4} = \frac{\text{?}}{4} = 4.4918 \times {10}^{- 11} kg \cdot {m}^{2}$$

$$\text{?} \text{indicates text missing or illegible when filed}$$

Max bending stress,

$$\text{?}$$

$$\text{?} \text{indicates text missing or illegible when filed}$$

According to Von Mises criterion,

$${S}_{F} = \frac{{σ}_{y}}{\sqrt{{σ}_{1}^{2} - {σ}_{1} {σ}_{2} + {σ}_{2}^{2}}} = \frac{{σ}_{y}}{{σ}_{M}} = \frac{250 \times {10}^{6}}{3.9489 \times {10}^{6}} = 63$$

As the bending stress is exactly reversed everytime the distal end 114 is deflected up and down by the same extent, mean stress=0. In calculating fatigue strength for 106 cycles,
C_{L}=1.0 (bending load)
C_{G}=1.0 (diameter<10 mm)
C_{S}=0.9 (commercially polished)
C_{T}=1.0 (room temperature)
C_{R}=0.897 (90% reliability)

$${S}_{n} = {S}_{n}^{′} {C}_{L} {C}_{O} {C}_{S} {C}_{T} {C}_{R} = 0.5 \times 400 \times {10}^{6} \times 1. \times 1. \times 0.9 \times 1. \times 0.897 = 161 MPa$$

As the safety factor is at a safe value of 63 and fatigue strength far exceeds the maximum bending stress experienced by the distal end of the inner tube 112, the inner tube 112 would be able to undergo cyclic loading up to 106 cycles without experiencing fatigue failure. The system of electronic components responsible for image capture and data transfer for live viewing purposes may also be an important aspect of the device 100.

The device 100 may have the capabilities of high-fidelity images/videos, basic treatment procedures, working channel and wireless data transmission. A good imaging quality contributes heavily to the basic functionality of any endoscope and wireless data transmission contributes to its portability, which can have potential for telemedicine. The resolution of images of the inner tube 112 is at least 640×480 pixels, its signal-to-noise ratio is approximately 20-40 dB, contrast-to-noise ratio of at least 2 and the angle of view of camera is in a range of 100°. The device 100 may also have live viewing and data capture capabilities via WiFi/Bluetooth to smartphone/tablets to allow for communication. In addition, the mass of the device 100 is lightweight, less than 300 g, for the clinician to handle and be fitted within the primary care setting.

FIG. 8 shows a flow chart illustrating a method 800 for manufacturing an endoscopy device, according to the example embodiments. The method comprises at step 802, providing a body having a handle portion and a housing portion, the housing portion having an outlet. At step 804, the method includes disposing a switching mechanism in the housing portion. At step 806, the method includes detachably coupling a rigid outer tube to the housing portion at the outlet. At step 808, the method includes disposing a flexible inner tube in the outer tube, wherein the switching mechanism is configured to retract or extend the inner tube outwardly through the outer tube. The method may further include inserting at least one guide wire inside the inner tube and coupling one end of the guide wire to the distal end of the inner tube and the other end of the guide wire to a control button, such that an activation of the button moves the distal end of the inner tube. The method of disposing the switching mechanism in the housing portion may comprise attaching a protrusion to the pulley and slidingly translating the protrusion to retract or extend the endoscope via a slot of the housing portion.

The endoscopy device 100 as described herein may not only serve general practitioners in the primary care setting to allow for quicker diagnosis and treatment, it could also serve to replace almost all the scopes currently used in the ENT specialist clinic. The device 100 may provide savings in the healthcare industry on multiple fronts. The device 100 would save ENT specialist clinics money in procuring and maintaining multiple pieces of costly equipment and clinicians need not be sent for multiple training for each piece of equipment and have a lower frequency of diagnosing and treating simple issues. Additionally, there are many prospects for use of this device 100 in the field of telemedicine, where professional advice and help from practiced healthcare professionals can be prescribed even outside of the clinical setting. The device 100 may be portable, which is especially pertinent in rural settings, where medical centers are scarce and medical attention is needed at off-the-grid locations.

The device 100 which includes flexible-rigid interchangeability, coupled with length adjustability of the insertion tube can suit a wider range of applications in different ENT regions for different use cases. Additionally, there is attainment of single-physician operation of the device 100 particularly in more complex therapeutic procedures that currently require an additional person's assistance in order to be performed. As the forceps and suction are the most commonly used therapeutic instruments in ENT, the device 100 with these instruments embedded within the handle would allow for greater convenience in the treatment of various ENT conditions.

While exemplary embodiments have been presented in the foregoing detailed description of the invention, it should be appreciated that a vast number of variations exist.

It should further be appreciated that the exemplary embodiments are only examples, and are not intended to limit the scope, applicability, operation, or configuration of the invention in any way. Rather, the foregoing detailed description will provide those skilled in the art with a convenient road map for implementing an exemplary embodiment of the invention, it being understood that various changes may be made in the function and arrangement of elements and method of operation described in an exemplary embodiment without departing from the scope of the invention as set forth in the appended claims.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

1. An endoscopy device comprising:
a body having a handle portion and a housing portion, the housing portion having an outlet;
a switching mechanism disposed in the housing portion;
a rigid outer tube detachably coupled to the housing portion at the outlet, and
a flexible inner tube extendable from the outer tube,
wherein the switching mechanism is configured to retract or extend the inner tube outwardly through the outer tube.

2. The endoscopy device according to claim 1, wherein the handle portion comprises means to control a distal end of the inner tube.

3. The endoscopy device according to claim 2, further comprising at least one guide wire inserted inside the inner tube and coupled to a control button, wherein an activation of the button moves the distal end of the inner tube.

4. The endoscopy device according to claim 1, wherein the switching mechanism comprises a pulley to retract or extend the inner tube.

5. The endoscopy device according to claim 4, wherein the switching mechanism comprises a protrusion attached to the pulley, and wherein the housing portion comprises a slot to slidingly translate the protrusion to retract or extend the inner tube.

6. The endoscopy device according to claim 1, wherein the handle portion further comprises an inlet, and wherein the inlet is configured to receive a medical instrument.

7. A method for manufacturing an endoscopy device, the method comprising:
providing a body having a handle portion and a housing portion, the housing portion having an outlet;
disposing a switching mechanism in the housing portion;
detachably coupling a rigid outer tube to the housing portion at the outlet, and
disposing a flexible inner tube in the outer tube,
wherein the switching mechanism is configured to retract or extend the inner tube outwardly through the outer tube.

8. The method according to claim 7, wherein the handle portion comprises means to control a distal end of the inner tube.

9. The method according to claim 8, further comprising inserting at least one guide wire inside the inner tube and coupling one end of the guide wire to the distal end of the inner tube and the other end of the guide wire to a control button, such that an activation of the button moves the distal end of the inner tube.

10. The method according to claim 7, wherein the switching mechanism comprises a pulley to retract or extend the inner tube.

11. The method according to claim 10, wherein disposing the switching mechanism in the housing portion comprises attaching a protrusion to the pulley and slidingly translating the protrusion to retract or extend the inner tube via a slot of the housing portion.

12. The method according to claim 7, wherein the handle portion further comprises an inlet, and wherein the inlet is configured to receive a medical instrument.
